(19) United States
(12) Patent Application Publication
(10) Pub. No.: US 2015/0025154 A1
(43) Pub. Date: Jan. 22, 2015

(54) OPHTHALMIC COMPOSITION

(71) Applicant: ROHTO PHARMACEUTICAL CO., LTD., Osaka (JP)

(72) Inventors: Takayuki MIYANO, Osaka (JP); Takahiro KUROSE, Osaka (JP)

(21) Appl. No.: 14/506,731

(22) Filed: Oct. 6, 2014

Related U.S. Application Data
(62) Division of application No. 13/775,644, filed on Feb. 25, 2013.
(60) Provisional application No. 61/638,716, filed on Apr. 26, 2012.

(30) Foreign Application Priority Data
Feb. 27, 2012 (JP) 2012-040675

(51) Int. Cl. A61K 47/10; A61K 9/00; A61K 47/14; A61K 9/06; A61K 47/44; A61K 31/121; A61K 9/08

(52) U.S. Cl. CPC A61K 47/10; A61K 31/121; A61K 9/0048; A61K 9/08; A61K 9/06; A61K 47/44; A61K 47/14
USPC 514/675

(57) ABSTRACT

An ophthalmic composition comprising geranylgeranylacetone and a fat-soluble antioxidant has an advantage that adsorption of geranylgeranylacetone to a wall of a container is remarkably reduced and thereby the loss of the geranylgeranylacetone content or decrease in the geranylgeranylacetone concentration in the composition is remarkably reduced. Adsorption of the fat-soluble antioxidant to a wall of a container is also reduced due to combination with geranylgeranylacetone.

### TECHNICAL FIELD

The present invention relates to an ophthalmic composition comprising geranylgeranylacetone.

### BACKGROUND ART

Teprenone (Eisai Co., Ltd.) is a mixture of (5E,9E,13E)-geranylgeranylacetone (hereinafter sometimes referred to as “all-trans form”) and (5Z,9E,13E)-geranylgeranylacetone (hereinafter sometimes referred to as “5Z-mono-cis form”) at a weight ratio of 3:2. Teprenone is widely used as an oral therapeutic agent for gastric ulcer.

The use of teprenone in the ophthalmic field has been suggested. For example, Patent Literature 1 teaches the use of teprenone as an active ingredient of a prophylactic or therapeutic agent for dry eye, eye strain, or eye dryness.

Patent Literature 2 discloses a clear eye drop consisting of teprenone, a phospholipid, a synthetic surfactant, and water.

However, an ophthalmic composition comprising geranylgeranylacetone has a problem that the geranylgeranylacetone content tends to be reduced during storage.

### CITATION LIST

### Patent Literature

Patent Literature 1: JP-8-133967 A

Patent Literature 2: JP-2000-319170 A

### SUMMARY OF INVENTION

### Technical Problem

An object of the present invention is to provide an ophthalmic composition comprising geranylgeranylacetone, the composition being characterized by that the loss of the geranylgeranylacetone content is reduced.

### Solution to Problem

The inventors conducted extensive research in order to solve the above problem and unexpectedly found that the addition of a fat-soluble antioxidant to an ophthalmic composition comprising geranylgeranylacetone (hereinafter sometimes referred to as “GGA”) remarkably reduces adsorption of GGA to a container wall, thereby remarkably reducing the loss of the GGA content of the ophthalmic composition.

The present invention has been completed based on the above finding and provides an ophthalmic composition as described below.

(1) An ophthalmic composition comprising geranylgeranylacetone and a fat-soluble antioxidant.

(2) The ophthalmic composition according to the above (1), wherein the fat-soluble antioxidant is a fat-soluble antioxidant other than tocopherol.

(3) The ophthalmic composition according to the above (1) or (2), wherein the fat-soluble antioxidant content is 0.00001 to 10% by weight relative to the total amount of the composition.

(4) The ophthalmic composition according to any of the above (1) to (3), wherein the geranylgeranylacetone content is 0.00001 to 10% by weight relative to the total amount of the composition.

(5) The ophthalmic composition according to any of the above (1) to (4), whose pH is from 6 to 8.

(6) The ophthalmic composition according to any of the above (1) to (5), which further comprises a phosphate buffering agent.

(7) The ophthalmic composition according to any of the above (1) to (6), which is in the form of a liquid, a fluid, a gel or a semi-solid.

(8) A method for reducing the loss of the geranylgeranylacetone content of a composition,

the method comprising the step of adding a fat-soluble antioxidant to an ophthalmic composition being held by an ophthalmic container and comprising geranylgeranylacetone, thereby reducing the loss of the geranylgeranylacetone content of the composition.

(9) A method for reducing adsorption of geranylgeranylacetone to a wall of an ophthalmic container, the method comprising the step of adding a fat-soluble antioxidant to an ophthalmic composition being held by an ophthalmic container and comprising geranylgeranylacetone, thereby reducing adsorption of geranylgeranylacetone to a wall of the ophthalmic container.

(10) Use of a combination of geranylgeranylacetone and a fat-soluble antioxidant for the production of an ophthalmic composition.

(11) Use, as an ophthalmic composition, of a composition comprising geranylgeranylacetone and a fat-soluble antioxidant.

### Advantageous Effects of Invention

The addition of a fat-soluble antioxidant to the ophthalmic composition of the present invention remarkably reduces adsorption of GGA to an ophthalmic container, thereby remarkably reducing the loss of the GGA content or decrease in the GGA concentration in the composition. Some kinds of materials for an ophthalmic container tend to adsorb a component of an ophthalmic composition. However, since the ophthalmic composition of the present invention comprises a fat-soluble antioxidant, adsorption of GGA is very little regardless of the material of the container.

Another advantage of the ophthalmic composition of the present invention is that GGA in the composition has good thermal and light stabilities due to the addition of a fat-soluble antioxidant.

Another advantage of the ophthalmic composition of the present invention is that adsorption of GGA to a contact lens is reduced due to the addition of a fat-soluble antioxidant.

Another advantage of the ophthalmic composition of the present invention is that adsorption of a fat-soluble antioxidant to the wall of an ophthalmic container is also reduced due to the presence of GGA.

### DESCRIPTION OF EMBODIMENTS

The present invention will be described in detail below.

The ophthalmic composition of the present invention is an ophthalmic composition comprising GGA and a fat-soluble antioxidant.

### Geranylgeranylacetone

### (1) Types of Geometric Isomers

GGA has eight geometric isomers. Specifically, the eight geometric isomers are:
(5E,9E,13E)-6,10,14,18-tetramethyl-5,9,13,17-nonadecatetrae n-2-one (5E,9E,13E GGA) (all-trans form),
(5Z,9E,13E)-6,10,14,18-tetramethyl-5,9,13,17-nonadecatetrae n-2-one (5Z,9E,13E GGA) (5Z-mono-cis form),
(5Z,9Z,13E)-6,10,14,18-tetramethyl-5,9,13,17-nonadecatetrae n-2-one (5Z,9Z,13E GGA) (13E-mono-trans form),
(5Z,9Z,13Z)-6,10,14,18-tetramethyl-5,9,13,17-nonadecatetrae n-2-one (5Z,9Z,13Z GGA) (all-cis form),
(5E,9Z,13E)-6,10,14,18-tetramethyl-5,9,13,17-nonadecatetrae n-2-one (5E,9Z,13E GGA) (9Z-mono-cis form),
(5E,9Z,13Z)-6,10,14,18-tetramethyl-5,9,13,17-nonadecatetrae n-2-one (5E,9Z,13Z GGA) (5E-mono-trans form),
(5E,9E,13Z)-6,10,14,18-tetramethyl-5,9,13,17-nonadecatetrae n-2-one (5E,9E,13Z GGA) (13Z-mono-cis form), and
(5Z,9E,13Z)-6,10,14,18-tetramethyl-5,9,13,17-nonadecatetrae n-2-one (5Z,9E,13Z GGA) (9E-mono-trans form).

These GGAs can be used alone or in any combination of two or more thereof according to the present invention. In cases where two or more of the GGAs are combined, the mixing ratio is not particularly limited.

Among the above GGAs, preferred are the all-trans form, the mono-cis forms (especially the 5Z-mono-cis form) and a mixture of the all-trans form and one of the mono-cis forms.

In cases where the GGA of the present invention is a mixture of the all-trans form and one of the mono-cis forms (especially the 5Z-mono-cis form), the all-trans form content of the mixture is preferably 80% by weight or more, more preferably 82% by weight or more, further more preferably 84% by weight or more, further more preferably 86% by weight or more, further more preferably 88% by weight or more, further more preferably 90% by weight or more, further more preferably 92% by weight or more, further more preferably 94% by weight or more, further more preferably 96% by weight, further more preferably 98% by weight or more. Especially preferably, the GGA consists of the all-trans form. When the all-trans form is in the above ranges, white turbidity at low temperature is reduced.

Also preferred GGA is a mixture of the all-trans form and one of the mono-cis forms (especially the 5Z-mono-cis form) with a very high mono-cis form (especially the 5Z-mono-cis form) ratio.

(2) All-trans form and 5Z-mono-cis form

(5E,9E,13E)-geranylgeranylacetone (the all-trans form) is a compound represented by the following structural formula:

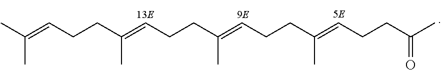 [SMILES: CC(=O)CC/C=C(\C)CC/C=C(\C)CC/C=C(\C)CCC=C(C)C]

The all-trans form can be purchased from, for example, Rionlon Development Co., Ltd.

The all-trans form can also be obtained through separating the all-trans form and the 5Z-mono-cis form of a marketed teprenone (Eisai Co., Ltd., Wako Pure Chemical Industries, Ltd., Yoshindo Inc., etc.) by, for example, silica gel chromatography using a mobile phase of n-hexane/ethyl acetate (9:1). The separation of the all-trans form and the 5Z-mono-cis form of a marketed teprenone can also be commissioned to, for example, KNC Laboratories Co., Ltd.

(5Z,9E,13E)-geranylgeranylacetone (the 5Z-mono-cis form) can also be obtained by the separation from a marketed teprenone. The 5Z-mono-cis form is a compound represented by the following structural formula:

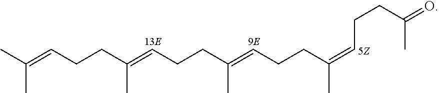 [SMILES: CC(=O)CC/C=C(/C)CC/C=C(\C)CC/C=C(\C)CCC=C(C)C]

The all-trans form can also be synthesized in accordance with a method described in, for example, Bull. Korean Chem. Soc., 2009, Vol. 30, No. 9, 215-217. This literature describes, for example, the method shown by the following synthesis scheme:

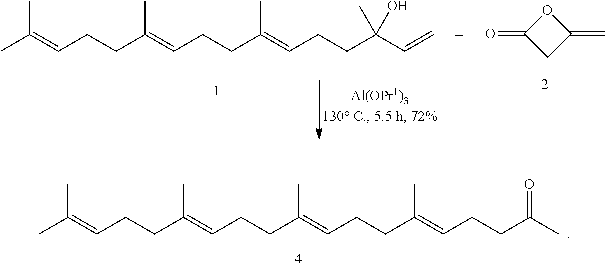

Specifically, in the above reaction formula, geranyllinalool 1 is mixed with Compound 2 and aluminum isopropoxide, and the mixture is gradually heated to 130° C. to allow the reaction to occur. After the completion of the reaction, the residue Compound 2 is removed and the reaction mixture is diluted with 5% sodium carbonate so that the residue aluminum propoxide is quenched. In this way, the all-trans form can be obtained. The obtained all-trans form is subsequently purified by, for example, silica gel chromatography using dichloromethane as an eluent.

(3) Mixtures of all-Trans Form and 5Z-mono-cis Form

Mixtures of the all-trans form and the 5Z-mono-cis form can be obtained by adding the all-trans form or the 5Z-mono-cis form to a marketed teprenone.

### (4) GGA Content

The GGA content of the ophthalmic composition is preferably 0.00001% by weight or more, more preferably 0.0001% by weight or more, further more preferably 0.001% by weight or more, relative to the total amount of the composition. The GGA content may be 0.01% by weight or more, 0.1% by weight or more, or 1% by weight or more. The GGA in the above ranges is sufficient to exert its pharmacological action.

The GGA content of the ophthalmic composition is preferably 10% by weight or less, more preferably 5% by weight or less, further more preferably 3% by weight or less, relative to the total amount of the composition. The ophthalmic composition comprising GGA in the above ranges allows clearer vision and hardly causes blurred vision.

The GGA content of the ophthalmic composition is, for example, about 0.00001 to 10% by weight, about 0.00001 to 5% by weight, about 0.00001 to 3% by weight, about 0.0001 to 10% by weight, about 0.0001 to 5% by weight, about 0.0001 to 3% by weight, about 0.001 to 10% by weight, about 0.001 to 5% by weight, about 0.001 to 3% by weight, about 0.01 to 10% by weight, about 0.01 to 5% by weight, about 0.01 to 3% by weight, about 0.1 to 10% by weight, about 0.1 to 5% by weight, about 0.1 to 3% by weight, about 1 to 10% by weight, about 1 to 5% by weight, or about 1 to 3% by weight, relative to the total amount of the composition.

### Fat-Soluble Antioxidant (Oil-Soluble Antioxidant)

Examples of the fat-soluble antioxidant include butyl group-containing phenols such as butylhydroxytoluene (BHT) and butylhydroxyanisole (BHA); nordihydroguaiaretic acid (NDGA); ascorbic acid esters such as ascorbyl palmitate, ascorbyl stearate, ascorbyl aminopropyl phosphate, ascorbyl tocopherol phosphate, ascorbic acid triphosphate, and ascorbyl palmitate phosphate; tocopherols such as α-tocopherol, β-tocopherol, γ-tocopherol, and 6-tocopherol; tocopherol derivatives such as tocopherol acetate, tocopherol nicotinate, and tocopherol succinate; gallic acid esters such as ethyl gallate, propyl gallate, octyl gallate, and dodecyl gallate; propyl gallate; 3-butyl-4-hydroxyquinolin-2-one; vegetable oils such as soybean oil, rapeseed oil, olive oil, and sesame oil; carotenoids such as lutein and astaxanthin; polyphenols such as anthocyanins, catechin, tannin, and curcumin; the vitamin A group including retinol, retinol esters (retinol acetate, retinol propionate, retinol butyrate, retinol octylate, retinol laurate, retinol stearate, retinol myristate, retinol oleate, retinol linolenate, retinol linoleate, retinol palmitate, etc.), retinal, retinal esters (retinal acetate, retinal propionate, retinal palmitate, etc.), retinoic acid, retinoic acid esters (methyl retinoate, ethyl retinoate, retinol retinoate, tocopheryl retinoate, etc.), dehydro forms of retinol, dehydro forms of retinal, dehydro forms of retinoic acid, provitamin A (α-carotene, β-carotene, γ-carotene, δ-carotene, lycopene, zeaxanthin, β-cryptoxanthin, echinenone, etc.), and vitamin A; CoQ10, etc. These compounds are marketed.

Among these, preferred are fat-soluble antioxidants other than tocopherol. Preferred are, for example, butyl group-containing phenols, NDGA, ascorbic acid esters, tocopherol derivatives, gallic acid esters, propylgallate, and 3-butyl-4-hydroxyquinolin-2-one, vegetable oils, and the vitamin A group. Among these, preferred are butyl group-containing phenols, tocopherol derivatives, vegetable oils, and the vitamin A group, more preferred are butyl group-containing phenols, vegetable oils, retinol, and retinol esters, and further more preferred are BHT, BHA, sesame oil, and retinol palmitate.

These fat-soluble antioxidants can be used alone or in combination of two or more thereof.

The fat-soluble antioxidant content of the ophthalmic composition is preferably 0.00001% by weight or more, more preferably 0.00005% by weight or more, further more preferably 0.0001% by weight or more, further more preferably 0.0005% by weight or more, relative to the total amount of the ophthalmic composition. The fat-soluble antioxidant in the above ranges is sufficient to exhibit the effects of reducing adsorption of GGA to a container wall (thereby reducing the loss of the GGA content), reducing adsorption of GGA to a contact lens, and improving the thermal and light stabilities of GGA.

The fat-soluble antioxidant content of the ophthalmic composition is preferably 10% by weight or less, more preferably 5% by weight or less, further more preferably 2% by weight or less, further more preferably 1% by weight or less, relative to the total amount of the composition. When the fat-soluble antioxidant is in the above ranges, the ophthalmic composition exhibits reduced eye irritancy.

The fat-soluble antioxidant content of the ophthalmic composition is, for example, about 0.00001 to 10% by weight, about 0.00001 to 5% by weight, about 0.00001 to 2% by weight, about 0.00001 to 1% by weight, about 0.00005 to 10% by weight, about 0.00005 to 5% by weight, about 0.00005 to 2% by weight, about 0.00005 to 1% by weight, about 0.0001 to 10% by weight, about 0.0001 to 5% by weight, about 0.0001 to 2% by weight, about 0.0001 to 1% by weight, about 0.0005 to 10% by weight, about 0.0005 to 5% by weight, about 0.0005 to 2% by weight, or about 0.0005 to 1% by weight, relative to the total amount of the ophthalmic composition.

The fat-soluble antioxidant content of the ophthalmic composition is preferably 0.0001 parts by weight or more, more preferably 0.001 parts by weight or more, further more preferably 0.005 parts by weight or more, further more preferably 0.01 parts by weight or more, relative to 1 part by weight of GGA. The fat-soluble antioxidant in the above ranges is sufficient to exhibit the effects of reducing adsorption of GGA to a container wall (thereby reducing the loss of the GGA content), reducing adsorption of GGA to a contact lens, and improving the thermal and light stabilities of GGA.

The fat-soluble antioxidant content of the ophthalmic composition is preferably 100 parts by weight or less, more preferably 50 parts by weight or less, further more preferably 10 parts by weight or less, further more preferably 5 parts by weight or less, relative to 1 part by weight of GGA. When the fat-soluble antioxidant is in the above ranges, the ophthalmic composition exhibits reduced eye irritancy.

The fat-soluble antioxidant content of the ophthalmic composition is, for example, about 0.0001 to 100 parts by weight, about 0.0001 to 50 parts by weight, about 0.0001 to 10 parts by weight, about 0.0001 to 5 parts by weight, about 0.001 to 100 parts by weight, about 0.001 to 50 parts by weight, about 0.001 to 10 parts by weight, about 0.001 to 5 parts by weight, about 0.005 to 100 parts by weight, about 0.005 to 50 parts by weight, about 0.005 to 10 parts by weight, about 0.005 to 5 parts by weight, about 0.01 to 100 parts by weight, about 0.01 to 50 parts by weight, about 0.01 to 10 parts by weight, or about 0.01 to 5 parts by weight, relative to 1 part by weight of GGA.

### Preparations

The form of the ophthalmic composition may be a liquid, a fluid, a gel or a semi-solid. Generally, components in a liquid or fluid composition tend to be adsorbed to a container wall. Hence, the present invention is suitably applied to a liquid or fluid ophthalmic composition. In addition, GGA in an aqueous composition tends to be adsorbed to a container wall and thus the present invention is also suitably applied to an aqueous composition.

The ophthalmic composition of the present invention can be formulated into any type of ophthalmic preparation. The dosage form employed in the present invention is, for example, an eye drop, an eye wash, a contact lens-wearing solution, a contact lens solution (e.g., a washing solution, a storage solution, a sterilizing solution, a multipurpose solution, a package solution, etc.), a preservative for a harvested ocular tissue (a cornea etc.) for transplantation, an irrigating solution for surgery, an ophthalmic ointment (e.g., a water-soluble ophthalmic ointment, an oil-soluble ophthalmic ointment, etc.), an intraocular injection (e.g., an intravitreal injection), etc. Among these, preferred are an eye drop, an eye wash, an ophthalmic ointment and an intraocular injection.

Preparation methods for an ophthalmic preparation are well known. An ophthalmic preparation can be prepared by mixing GGA with a pharmaceutically acceptable base or carrier, and as needed a pharmaceutically acceptable additive for an ophthalmic preparation and another active ingredient (a physiologically or pharmacologically active component).

### <Bases or Carriers>

Examples of the base or carrier include water; an aqueous solvent such as a polar solvent; a polyalcohol; a vegetable oil; and an oily base. Examples of the base or carrier for an intraocular injection include water for injection and physiological saline.

These bases or carriers can be used alone or in combination of two or more thereof.

### <Additives>

Examples of the additive include a surfactant, a flavor or cooling agent, an antiseptic, a bactericide or antibacterial agent, a pH adjusting agent, a tonicity agent, a chelating agent, a buffering agent, a stabilizer, another antioxidant, and a thickening agent. An intraocular injection may contain a solubilizing agent, a suspending agent, a tonicity agent, a buffering agent, a soothing agent, a stabilizer, and an antiseptic.

These additives can be used alone or in combination of two or more thereof.

The additives will be exemplified below.

Surfactants: for example, nonionic surfactants such as polyoxyethylene (hereinafter sometimes referred to as “POE”)-polyoxypropylene (hereinafter sometimes referred to as “POP”) block copolymers (e.g., poloxamer 407, poloxamer 235, poloxamer 188), ethylenediamine POE-POP block copolymer adducts (e.g., poloxamine), POE sorbitan fatty acid esters (e.g., polysorbate 20, polysorbate 60, polysorbate 80 (TO-10 etc.)), POE hydrogenated castor oils (e.g., POE (60) hydrogenated castor oil (HCO-60 etc.)), POE castor oils, POE alkyl ethers (e.g., polyoxyethylene (9) lauryl ether, polyoxyethylene (20) polyoxypropylene (4) cetyl ether), and polyoxyl stearate;

amphoteric surfactants such as glycine-type amphoteric surfactants (e.g., alkyl diaminoethyl glycine, alkyl polyaminoethyl glycine), betaine-type amphoteric surfactants (e.g., lauryldimethylaminoacetic betaine, imidazolinium betaine);
cationic surfactants such as alkyl quaternary ammonium salts (e.g., benzalkonium chloride, benzethonium chloride); etc.

The numbers in the parentheses represent the molar number of added POE or POP.

Flavors or cooling agents: for example, camphor, borneol, terpenes (these may be in the d-form, l-form, or dl-form); essential oils such as mentha water, eucalyptus oil, bergamot oil, anethole, eugenol, geraniol, menthol, limonene, mentha oil, peppermint oil, rose oil, etc.

Antiseptics, bactericides, or antibacterial agents: for example, polidronium chloride, alkyldiaminoethylglycine hydrochloride, sodium benzoate, ethanol, benzalkonium chloride, benzethonium chloride, chlorhexidine gluconate, chlorobutanol, sorbic acid, potassium sorbate, sodium dehydroacetate, methyl paraoxybenzoate, ethyl paraoxybenzoate, propyl paraoxybenzoate, butyl paraoxybenzoate, oxyquinoline sulfate, phenethyl alcohol, benzyl alcohol, biguanide compounds (in particular, polyhexamethylene biguanide or its hydrochloride etc.), Glokill (Rhodia Ltd.), etc.

pH adjusting agents: for example, hydrochloric acid, sodium hydroxide, potassium hydroxide, calcium hydroxide, magnesium hydroxide, triethanolamine, monoethanolamine, diisopropanolamine, sulfuric acid, phosphoric acid, etc.

Tonicity agents: for example, sodium bisulfite, sodium sulfite, potassium chloride, calcium chloride, sodium chloride, magnesium chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, glycerin, propylene glycol, etc.

Chelating agents: for example, ascorbic acid, edetic acid tetrasodium, sodium edetate, citric acid, etc.

Buffering agents: for example, phosphate buffering agents; citrate buffering agents such as citric acid and sodium citrate; acetate buffering agents such as acetic acid, potassium acetate, and sodium acetate; carbonate buffering agents such as sodium bicarbonate and sodium carbonate; borate buffering agents such as boric acid and borax; amino acid buffering agents such as taurine, aspartic acid and its salts (e.g., potassium salts etc.), and ε-aminocaproic acid; etc.

Among the above, phosphate buffering agents are preferred for pH adjustment and the use of a phosphate buffering agent reduces adsorption of GGA to a container wall, thereby further effectively reducing the loss of the GGA content of the composition. The use of a phosphate buffering agent also further effectively reduces adsorption of GGA to a contact lens, and further improves the thermal and light stabilities. The use of a phosphate buffering agent also provides the effect of reducing white turbidity during storage at low temperature.

The phosphate buffering agents can be used alone or in combination of two or more thereof.

The phosphate buffering agent is not particularly limited and examples thereof include phosphoric acid; alkali metal phosphates such as disodium hydrogen phosphate, sodium dihydrogen phosphate, trisodium phosphate, dipotassium hydrogen phosphate, potassium dihydrogen phosphate, and tripotassium phosphate; alkaline earth metal phosphates such as calcium phosphate, calcium hydrogen phosphate, calcium dihydrogen phosphate, monomagnesium phosphate, dimagnesium phosphate (magnesium hydrogen phosphate), and trimagnesium phosphate; and ammonium phosphates such as diammonium hydrogen phosphate and ammonium dihydrogen phosphate. The phosphate buffering agent may be an anhydride or hydrate.

Among the above, preferably at least one selected from the group consisting of phosphoric acid and alkali metal phosphates is used, and more preferably at least one selected from the group consisting of phosphoric acid and sodium phosphates is used.

Preferred combinations of phosphate buffering agents are, for example, a combination of phosphoric acid, disodium hydrogen phosphate, sodium dihydrogen phosphate, and trisodium phosphate; a combination of phosphoric acid, disodium hydrogen phosphate, and sodium dihydrogen phosphate; a combination of phosphoric acid, disodium hydrogen phosphate, and trisodium phosphate; a combination of phosphoric acid, sodium dihydrogen phosphate, and trisodium phosphate; a combination of disodium hydrogen phosphate, sodium dihydrogen phosphate, and trisodium phosphate; a combination of phosphoric acid and disodium hydrogen phosphate; a combination of phosphoric acid and sodium dihydrogen phosphate; a combination of phosphoric acid and trisodium phosphate; a combination of disodium hydrogen phosphate and sodium dihydrogen phosphate; a combination of disodium hydrogen phosphate and trisodium phosphate; and a combination of sodium dihydrogen phosphate and trisodium phosphate.

Among these, preferred are a combination of phosphoric acid, disodium hydrogen phosphate, and sodium dihydrogen phosphate; a combination of phosphoric acid and disodium hydrogen phosphate; a combination of phosphoric acid and sodium dihydrogen phosphate; and a combination of disodium hydrogen phosphate and sodium dihydrogen phosphate. More preferred is a combination of disodium hydrogen phosphate and sodium dihydrogen phosphate.

The phosphate buffering agent content expressed in terms of a corresponding anhydride is preferably 0.001% by weight or more, more preferably 0.005% by weight or more, further more preferably 0.01% by weight or more, further more preferably 0.05% by weight or more, relative to the total amount of the composition. The phosphate buffering agent in the above ranges is sufficient to exhibit the effects of stabilizing GGA, reducing white turbidity at low temperature, and reducing adsorption of GGA to a container wall or a contact lens.

The phosphate buffering agent content expressed in terms of a corresponding anhydride is preferably 10% by weight or less, more preferably 7% by weight or less, furthermore preferably 5% by weight or less, further more preferably 3% by weight or less, relative to the total amount of the composition. When GGA is in the above ranges, the ophthalmic composition exhibits reduced eye irritancy.

The phosphate buffering agent content expressed in terms of a corresponding anhydride is, for example, about 0.001 to 10% by weight, about 0.001 to 7% by weight, about 0.001 to 5% by weight, about 0.001 to 3% by weight, about 0.005 to 10% by weight, about 0.005 to 7% by weight, about 0.005 to 5% by weight, about 0.005 to 3% by weight, about 0.01 to 10% by weight, about 0.01 to 7% by weight, about 0.01 to 5% by weight, about 0.01 to 3% by weight, about 0.05 to 10% by weight, about 0.05 to 7% by weight, about 0.05 to 5% by weight, or about 0.05 to 3% by weight, relative to the total amount of the ophthalmic composition.

The phosphate buffering agent content expressed in terms of a corresponding anhydride is preferably 0.0005 parts by weight or more, more preferably 0.001 parts by weight or more, further more preferably 0.005 parts by weight or more, further more preferably 0.01 parts by weight or more, relative to 1 part by weight of GGA. The phosphate buffering agent in the above ranges is sufficient to exhibit the effects of stabilizing GGA, reducing white turbidity at low temperature, and reducing adsorption of GGA to a container wall or a contact lens.

The phosphate buffering agent content expressed in terms of a corresponding anhydride is preferably 5000 parts by weight or less, more preferably 1000 parts by weight or less, further more preferably 500 parts by weight or less, further more preferably 200 parts by weight or less, relative to 1 part by weight of GGA. When the phosphate buffering agent is in the above ranges, the ophthalmic composition exhibits reduced eye irritancy.

The phosphate buffering agent content expressed in terms of a corresponding anhydride is, for example, about 0.0005 to 5000 parts by weight, about 0.0005 to 1000 parts by weight, about 0.0005 to 500 parts by weight, about 0.0005 to 200 parts by weight, about 0.001 to 5000 parts by weight, about 0.001 to 1000 parts by weight, about 0.001 to 500 parts by weight, about 0.001 to 200 parts by weight, about 0.005 to 5000 parts by weight, about 0.005 to 1000 parts by weight, about 0.005 to 500 parts by weight, about 0.005 to 200 parts by weight, about 0.01 to 5000 parts by weight, about 0.01 to 1000 parts by weight, about 0.01 to 500 parts by weight, or about 0.01 to 200 parts by weight, relative to 1 part by weight of GGA.

Stabilizers: for example, trometamol, sodium formaldehyde sulfoxylate (rongalit), tocopherol, sodium pyrosulfite, monoethanolamine, aluminum monostearate, glyceryl monostearate, etc.

Other antioxidants: for example, ascorbic acid, ascorbic acid derivatives (ascorbic acid-2-sulfate disodium salts, sodium ascorbate, ascorbic acid-2-magnesium phosphate, ascorbic acid-2-sodium phosphate, etc.), sodium bisulfite, sodium sulfite, sodium thiosulfate, etc.

Thickening agents: for example, guar gum; hydroxypropyl guar gum; high molecular cellulose compounds such as methylcellulose, ethylcellulose, hydroxypropyl methylcellulose, hydroxyethyl cellulose, and carboxymethyl cellulose sodium; gum arabic; karaya gum; xanthan gum; agar-agar; alginic acid; α-cyclodextrin; dextrin; dextran; heparin; heparinoid; heparin sulfate; heparan sulfate; hyaluronic acid; hyaluronates (sodium salts etc.); sodium chondroitin sulfate; starch; chitin and its derivatives; chitosan and its derivatives; carrageenan; sorbitol; high molecular polyvinyl compounds such as polyvinylpyrrolidone, polyvinyl alcohol, and polyvinyl methacrylate; carboxy vinyl polymers such as alkali metal polyacrylates (sodium salts, potassium salts, etc.), amine polyacrylates (monoethanolamine salts, diethanolamine salts, triethanolamine salts, etc.), and ammonium polyacrylates; casein; gelatin; collagen; pectin; elastin; ceramide; liquid paraffin; glycerin; polyethylene glycol; macrogol; polyethyleneimine alginates (sodium salts etc.); alginate esters (propylene glycol esters etc.); powdered tragacanth; triisopropanolamine; etc.

### <Other Pharmacologically or Physiologically Active Components>

Pharmacologically or physiologically active components other than GGA can be used alone or in combination of two or more thereof.

Examples of the pharmacologically or physiologically active components include prophylactic or therapeutic components for a retinal disease, nerve growth factors, decongestants, drugs for restoring extraocular muscle function, anti-inflammatory drugs or astringent drugs, antihistaminics or antiallergics, vitamins, amino acids, antibacterial drugs or bactericides, sugars, high molecular compounds, celluloses or their derivatives, local anesthetics, etc. These components will be exemplified below.

Prophylactic or therapeutic components for a retinal disease: for example, prostaglandin F2α derivatives such as prost drugs (latanoprost, travoprost, tafluprost, etc.), prostamide drugs (bimatoprost etc.) and prostone drugs (isopropyl unoprostone); sympatholytic drugs such as β-blockers (timolol maleate, gel-forming timolol, carteolol hydrochloride, gel-forming carteolol, etc.), β1-blockers (betaxolol hydrochloride etc.), αβ-blockers (levobunolol hydrochloride, nipradilol, bunazosin hydrochloride, etc.) and α2 blockers (brimonidine tartrate); parasympathomimetic drugs such as pilocarpine hydrochloride and distigmine bromide; sympathomimetic drugs such as epinephrine, epinephrine hydrogen tartrate and dipivefrin hydrochloride; carbonic anhydrase inhibitors such as dorzolamide hydrochloride and brinzolamide; specific inhibitors to ROCK (Rho-associated coiled coil forming protein kinase) such as SNJ-1656 and K-115; calcium antagonists such as lomerizine hydrochloride; EP2 agonists such as DE-117; adenosine A2a receptor agonists such as OPA-6566; therapeutic agents for age-related macular degeneration such as VEGF aptamers (pegaptanib sodium) and VEGF inhibitors (ranibizumab, bevacizumab); etc.

Nerve growth factors: for example, nerve growth factor (NGF), brain-derived nerve growth factor (BDNF), glial cell line-derived neurotrophic factor (GDNF), etc.

Since nutritional factors including nerve growth factors are contained in serum, it is possible to add serum from a patient to a preparation for the patient.

Decongestants: for example, α-adrenergic agonists such as epinephrine, epinephrine hydrochloride, ephedrine hydrochloride, oxymetazoline hydrochloride, tetrahydrozoline hydrochloride, naphazoline hydrochloride, phenylephrine hydrochloride, methylephedrine hydrochloride, epinephrine hydrogen tartrate, naphazoline nitrate, etc. These may be in the d-form, l-form, or dl-form.

Drugs for restoring extraocular muscle function: for example, cholinesterase inhibitors having an active center similar to that of acetylcholine, such as neostigmine methylsulfate, tropicamide, helenien, atropine sulfate, etc.

Anti-inflammatory drugs or astringent drugs: for example, zinc sulfate, zinc lactate, allantoin, ε-aminocaproic acid, indomethacin, lysozyme chloride, silver nitrate, pranoprofen, azulene sulfonate sodium, dipotassium glycyrrhizinate, diammonium glycyrrhizinate, diclofenac sodium, bromfenac sodium, berberine chloride, berberine sulfate, etc.

Antihistaminics or antiallergics: for example, acitazanolast, diphenhydramine or its salts (hydrochloride etc.), chlorpheniramine maleate, ketotifen fumarate, levocabastine or its salts (hydrochloride etc.), amlexanox, ibudilast, tazanolast, tranilast, oxatomide, suplatast or its salts (tosilate etc.), sodium cromoglicate, pemirolast potassium, etc.

Vitamins: for example, pyridoxine hydrochloride, flavin adenine dinucleotide sodium, pyridoxal phosphate, cyanocobalamin, panthenol, calcium pantothenate, sodium pantothenate, ascorbic acid, tocopherol calcium succinate, ubiquinone derivatives, etc.

Amino acids: for example, aminoethylsulfonic acid (taurine), glutamic acid, creatinine, sodium aspartate, potassium aspartate, magnesium aspartate, magnesium potassium aspartate, sodium glutamate, magnesium glutamate, ε-aminocaproic acid, glycine, alanine, arginine, lysine, γ-aminobutyric acid, γ-aminovaleric acid, sodium chondroitin sulfate, etc. These may be in the d-form, l-form, or dl-form.

Antibacterial drugs or bactericides: for example, alkylpolyaminoethylglycine, chloramphenicol, sulfamethoxazole, sulfisoxazole, sulfamethoxazole sodium, sulfisoxazole diethanolamine, sulfisoxazole monoethanolamine, sulfisomezole sodium, sulfisomidine sodium, ofloxacin, norfloxacin, levofloxacin, lomefloxacin hydrochloride, acyclovir, etc.

Sugars: for example, monosaccharides, disaccharide, in particular, glucose, maltose, trehalose, sucrose, cyclodextrin, xylitol, sorbitol, mannitol, etc.

High molecular compounds: for example, alginic acid, sodium alginate, dextrin, dextran, pectin, hyaluronic acid, chondroitin sulfate, (completely or partially saponified) polyvinyl alcohol, polyvinylpyrrolidone, carboxy vinyl polymers, macrogol, pharmaceutically acceptable salts thereof, etc.

Celluloses or their derivatives: for example, ethylcellulose, hydroxyethyl cellulose, hydroxypropyl cellulose, hydroxypropyl methylcellulose, methylcellulose, carboxymethyl cellulose, carboxymethylcellulose sodium, carboxyethyl cellulose, nitrocellulose, etc.

Local anesthetics: for example, chlorobutanol, procaine hydrochloride, lidocaine hydrochloride, etc.

pH

The pH of the ophthalmic preparation is preferably 4 or higher, more preferably 5.5 or higher, further more preferably 6 or higher, further more preferably 6.5 or higher. When the ophthalmic preparation has a pH value in the above ranges, adsorption to a container wall is further effectively reduced and adsorption of GGA to a contact lens is also reduced. The preparation having a pH value in the above ranges is excellent in stability against light, heat, low temperature, etc.

The pH of the ophthalmic preparation is preferably 9 or lower, more preferably 8.5 or lower, further more preferably 8 or lower, further more preferably 7.5 or lower. The ophthalmic preparation having a pH value in the above ranges exhibits reduced eye irritancy.

### Usage

The usage of the ophthalmic composition of the present invention varies depending on its dosage form and the route of administration is appropriately selected in accordance with the dosage form.

For example, when the composition of the present invention is an eye drop, the eye drop comprising GGA in the above concentration ranges is instilled, for example, about 1 to 5 times a day, preferably about 1 to 3 times a day, in an amount of about 1 to 2 drops each time.

When the composition of the present invention is an eye wash, eye washing is performed, for example, about 1 to 10 times a day, preferably about 1 to 5 times a day, each time using about to 20 mL of the eye wash comprising GGA in the above concentration ranges.

When the composition of the present invention is an ophthalmic ointment, the ophthalmic ointment comprising GGA in the above concentration ranges is applied to the eye, for example, about 1 to 5 times a day, preferably about 1 to 3 times a day, in an amount of about 0.001 to 5 g each time.

When the composition of the present invention is an intraocular injection, the intraocular injection comprising GGA in the above concentration ranges is injected, for example, about 1 to 3 times per day to 14 days, preferably once per day to 14 days, in an amount of about 0.005 to 1 mL each time.

When the composition of the present invention is a contact lens solution (a washing solution, a storage solution, a sterilizing solution, a multipurpose solution, package solution, etc.), a preservative for a harvested ocular tissue (a cornea etc.) for transplantation, or an irrigating solution for surgery, such a composition comprising GGA in the above concentration ranges is used in a usual dosage and regimen of such a type of preparation.

When the composition of the present invention is a sustained-release contact lens preparation, the contact lens comprising GGA in the above amount is replaced with a fresh one, for example, about 1 to 3 times per day to 14 days, preferably once per day to 14 days.

When the composition of the present invention is a sustained-release intraocular implant, about 1 to 14 days after the implantation of the implant comprising GGA in the above amount, a fresh one is implanted as needed.

The administration period varies depending on the type and stage of the disease, the age, weight, and sex of the patient, the route of administration, etc., and can be selected as appropriate, for example, from the range from about one day to 30 years. When the retinal protective action exhibited by the ophthalmic composition of the present invention suppresses the progress of a retinal disease, the administration can be further continued.

### Others

The present invention includes

a method for reducing the loss of the GGA content, the method comprising adding a fat-soluble antioxidant to an ophthalmic composition being held by an ophthalmic container and comprising GGA,
a method for reducing adsorption of GGA to a wall of an ophthalmic container, the method comprising adding a fat-soluble antioxidant to an ophthalmic composition being held by an ophthalmic container and comprising GGA, and
a method for reducing adsorption of a fat-soluble antioxidant to a wall of an ophthalmic container, the method comprising adding GGA to an ophthalmic composition held by an ophthalmic container and comprising a fat-soluble antioxidant.

In these methods of the present invention, the type and dosage of GGA, the type and dosage of the fat-soluble antioxidant, the type, properties, and other components of the ophthalmic composition, etc. are as described for the ophthalmic composition of the present invention.

The material of the ophthalmic container is not particularly limited as long as the material is usually used for an ophthalmic container. Examples of the ophthalmic container include an ophthalmic container whose surface in contact with the ophthalmic composition is at least partially or wholly made of at least one material selected from the group consisting of a polyolefin, an acrylic acid resin, a terephthalic acid ester, a 2,6-naphthalene dicarboxylic acid ester, a polycarbonate, a polymethylterpene, a fluorine resin, a polyvinyl chloride, a polyamide, an ABS resin, an AS resin, a polyacetal, a modified polyphenylene ether, a polyarylate, a polysulfone, a polyimide, a cellulose acetate, a hydrocarbon optionally substituted with a halogen atom, a polystyrene, a polybutylene succinate, an aluminum and a glass.

Examples of the polyolefin include polyethylenes (including high density polyethylene, low density polyethylene, ultra low density polyethylene, linear low density polyethylene, ultra high molecular weight polyethylene, etc.), polypropylenes (including isotactic polypropylene, syndiotactic polypropylene, atactic polypropylene, etc.), ethylene-propylene copolymers, etc.

Examples of the acrylic acid resin include acrylic acid esters such as methyl acrylate, methacrylic acid esters such as methyl methacrylate, cyclohexyl methacrylate and t-butyl cyclohexyl methacrylate, etc.

Examples of the terephthalic acid ester include polyethylene terephthalate, polytrimethylene terephthalate, polybutylene terephthalate, etc.

Examples of the 2,6-naphthalene dicarboxylic acid ester include polyethylene naphthalate, polybutylene naphthalate, etc.

Examples of the fluorine resin include fluorine-substituted polyethylenes (polytetrafluoroethylene, polychlorotrifluoroethylene, etc.), polyvinylidene fluoride, polyvinyl fluoride, perfluoroalkoxy fluorine resins, tetrafluoroethylene-hexafluoropropylene copolymers, ethylene-tetrafluoroethylene copolymers, ethylene-chlorotrifluoroethylene copolymers, etc.

Examples of the polyamide include nylon etc.

Examples of the polyacetal include polyacetals consisting of oxymethylene units, polyacetals containing oxyethylene units, etc.

Examples of the modified polyphenylene ether include polystyrene-modified polyphenylene ether etc.

Examples of the polyarylate include amorphous polyarylate etc.

Examples of the polyimide include aromatic polyimides such as the one obtained by polymerizing pyromellitic dianhydride and 4,4′-diaminodiphenyl ether.

Examples of the cellulose acetate include cellulose diacetate, cellulose triacetate, etc.

Examples of the hydrocarbon optionally substituted with a halogen atom include hydrocarbons such as methane, ethane, propane, butane, ethylene, propylene, 1-butene, 2-butene and 1,3-butadiene; hydrocarbons substituted with a fluorine atom; hydrocarbons substituted with a chlorine atom; hydrocarbons substituted with a bromine atom; hydrocarbons substituted with an iodine atom; etc.

### Examples

The present invention will be described in more detail below with reference to Examples, but the present invention is not limited thereto.

### (1) Preparation of Geranylgeranylacetone

Marketed teprenone (all-trans form: 5Z-mono-cis form=6:4 (weight ratio)) (Wako Pure Chemical Industries, Ltd.) was purchased and the all-trans form was separated and purified by silica gel chromatography.

The above preparative purification was carried out using silica gel (PSQ60B, Fuji Silysia Chemical Ltd.) filled in a glass tube and a mobile phase of n-hexane/ethyl acetate (9:1). After the separation, each fraction was concentrated and dried under reduced pressure and the degree of purification and structure of the all-trans form were determined by GC and ¹H-NMR (solvent: deuterated chloroform; internal standard: tetramethylsilane) (about 20% yield).

### <GC Measurement Conditions>

Column: DB-1 (J&W Scientific, 0.53 mm×30 m, film thickness of 1.5 μm)
Column temperature: elevated at a rate of 5° C./minute from 200° C. to 300° C. (10 minutes)
Vaporizing chamber temperature: 280° C.
Detector temperature: 280° C.
Carrier gas: helium
Hydrogen pressure: 60 kPa
Air pressure: 50 kPa
Makeup gas pressure: 75 kPa (nitrogen gas)
Total flow: 41 mL/min
Column flow: 6.52 mL/min
Linear velocity: 58.3 cm/sec
Split ratio: 5:1
Injection volume: 1 μL of 0.1 g/100 mL sample (in ethanol)

### (2) Measurement Method for GGA Concentration

In accordance with the measurement conditions for the elution test described in PFSB/ELD Notification No. 0412007 “teprenone 100 mg/g fine granule”, the GGA concentration of each eye drop was determined from the area value of the 5Z-mono-cis form (Ac) and the area value of the all-trans form (At) using Japanese pharmacopoeia “teprenone reference standard (all-trans form:5Z-mono-cis form=about 6:4 (weight ratio), Pharmaceutical and Medical Device Regulatory Science Society of Japan)” or teprenone (Wako Pure Chemical Industries) as a reference standard under the HPLC measurement conditions described below. For the eye drop containing teprenone (all-trans form:5Z-mono-cis form=3:2 (weight ratio)), the GGA content was calculated by summing the amounts of the all-trans form and the 5Z-mono-cis form.

### <HPLC Measurement Conditions>

Detector: ultraviolet absorption spectrometer (measurement wavelength: 210 nm)
Column: YMC-Pack ODS-A (inner diameter: 4.6 mm, length: 15 cm, particle diameter: 3 μm)
Column temperature: 30° C.
Mobile phase: 90% acetonitrile solution
Flow rate: 1.2 to 1.3 mL/min (the 5Z-mono-cis form and the all-trans form are eluted in this order.)
Injection volume: 5 μL of 0.05 g/100 mL sample

### (3) Test for Residual Ratio of GGA No. 1

Eye drops containing the marketed teprenone or GGA consisting of the all-trans form purified by the above method were prepared as follows. The constitutions of the eye drops are shown in Tables 1 to 3 below.

Specifically, to a surfactant (polysorbate 80 and POE castor oil) warmed to 65° C., teprenone or the all-trans form, and BHT for Examples, were added and dissolved under stirring in a hot water bath at 65° C. for 2 minutes. Water at 65° C. was added and each buffer was added under stirring to give a homogeneous solution. The pH and osmotic pressure were adjusted with hydrochloric acid and/or sodium hydroxide. This resulting solution was filtered through a membrane filter with a pore size of 0.2 μM (bottle top filter, Thermo Fisher Scientific) to give a clear sterile eye drop. Before the preparation of the sterile eye drops, it was confirmed by HPLC described later that adsorption of GGA to instruments etc., which leads to the loss of the GGA content, did not occur during the preparation procedure.

A polyethylene terephthalate container (8 mL) (the container for Rohto Dryaid EX, Rohto Pharmaceutical) was completely filled with each of the eye drops in an aseptic manner. A 10 mL clear glass container (Vial with Screw Cap S-3, Nichiden-Rika Glass) was also completely filled with each of the eye drops (so that no air space remained) in an aseptic manner. For each of the eye drops, the stability test was performed by leaving them to stand in the upright position at 40° C. or 50° C. for 20 days, or 60° C. for 10 days. The teprenone or all-trans form content (g/100 mL) in each of the eye drops was quantified under the HPLC conditions described above immediately after the production and after being left to stand for a predetermined period of time, and the residual ratio (%) was calculated.

Residual ratio (%)=100×[teprenone or all-trans form content after being left to stand for predetermined period of time (g/100 mL)/teprenone or all-trans form content (g/100 mL)]

The results are shown in Tables 1 to 3.

TABLE 1

| | | | | | Com- | Com- |
|---|---|---|---|---|---|---|
| | | | | | parative | parative |
| | | | Exam- | Exam- | Exam- | Exam- |
| g/100 mL | | | ple 1 | ple 2 | ple 1 | ple 2 |
| All-trans form | | | 0.05 | — | 0.05 | — |
| All-trans form:5Z-mono-cis form | | | — | 0.05 | — | 0.05 |
| weight ratio (6:4) | | | | | | |
| Sodium dihydrogen phosphate | | | 2.00 | 2.00 | 2.00 | 2.00 |
| dihydrate | | | | | | |
| Disodium hydrogen phosphate | | | 0.40 | 0.40 | 0.40 | 0.40 |
| dodecahydrate | | | | | | |
| POE castor oil | | | 0.02 | 0.02 | 0.02 | 0.02 |
| Polysorbate 80 | | | 0.50 | 0.50 | 0.50 | 0.50 |
| BHT | | | 0.005 | 0.005 | — | — |
| Hydrochloric acid | | | q.s. | q.s. | q.s. | q.s. |
| Sodium hydroxide | | | q.s. | q.s. | q.s. | q.s. |
| Purified water | | | q.s. | q.s. | q.s. | q.s. |
| pH | | | 5.7 | 5.7 | 5.7 | 5.7 |
| Osmotic pressure mOsm | | | 270 | 270 | 270 | 270 |
| Residual | 40° C. 20 days | Glass | 99.9 | 99.5 | 99.9 | 99.5 |
| ratio | | PET | 100.2 | 99.9 | 98.4 | 97.3 |
| (%) | 50° C. 20 days | Glass | 99.2 | 99.2 | 97.9 | 96.5 |
| | | PET | 101.7 | 101.4 | 91.9 | 90.2 |
| | 60° C. 10 days | Glass | 102.2 | 102.2 | 97.9 | 96.0 |
| | | PET | 100.6 | 100.5 | 84.8 | 85.6 |

TABLE 2

| | | | | | Com- | Com- |
|---|---|---|---|---|---|---|
| | | | | | parative | parative |
| | | | Exam- | Exam- | Exam- | Exam- |
| g/100 mL | | | ple 3 | ple 4 | ple 3 | ple 4 |
| All-trans form | | | 0.05 | — | 0.05 | — |
| All-trans form:5Z-mono-cis form | | | — | 0.05 | — | 0.05 |
| weight ratio (6:4) | | | | | | |
| Sodium dihydrogen phosphate | | | 1.40 | 1.40 | 1.40 | 1.40 |
| dihydrate | | | | | | |
| Disodium hydrogen phosphate | | | 1.40 | 1.40 | 1.40 | 1.40 |
| dodecahydrate | | | | | | |
| POE castor oil | | | 0.02 | 0.02 | 0.02 | 0.02 |
| Polysorbate 80 | | | 0.50 | 0.50 | 0.50 | 0.50 |
| BHT | | | 0.005 | 0.005 | — | — |
| Hydrochloric acid | | | q.s. | q.s. | q.s. | q.s. |
| Sodium hydroxide | | | q.s. | q.s. | q.s. | q.s. |
| Purified water | | | q.s. | q.s. | q.s. | q.s. |
| pH | | | 6.5 | 6.5 | 6.5 | 6.5 |
| Osmotic pressure mOsm | | | 270 | 270 | 270 | 270 |
| Residual | 40° C. 20 days | Glass | 100.3 | 100.4 | 99.7 | 99.9 |
| ratio | | PET | 100.7 | 100.4 | 98.0 | 98.3 |
| (%) | 50° C. 20 days | Glass | 100.1 | 100.0 | 99.0 | 97.5 |
| | | PET | 102.5 | 102.3 | 94.3 | 94.6 |
| | 60° C. 10 days | Glass | 102.5 | 102.5 | 101.3 | 100.5 |
| | | PET | 100.6 | 100.8 | 90.7 | 91.8 |

TABLE 3

| | | | | | Com- | Com- |
|---|---|---|---|---|---|---|
| | | | | | parative | parative |
| | | | Exam- | Exam- | Exam- | Exam- |
| g/100 mL | | | ple 5 | ple 6 | ple 5 | ple 6 |
| All-trans form | | | 0.05 | — | 0.05 | — |
| All-trans form:5Z-mono-cis form | | | — | 0.05 | — | 0.05 |
| weight ratio (6:4) | | | | | | |
| Sodium dihydrogen phosphate | | | 0.30 | 0.30 | 0.30 | 0.30 |
| dihydrate | | | | | | |
| Disodium hydrogen phosphate | | | 3.20 | 3.20 | 3.20 | 3.20 |
| dodecahydrate | | | | | | |
| POE castor oil | | | 0.02 | 0.02 | 0.02 | 0.02 |
| Polysorbate 80 | | | 0.50 | 0.50 | 0.50 | 0.50 |
| BHT | | | 0.005 | 0.005 | — | — |
| Hydrochloric acid | | | q.s. | q.s. | q.s. | q.s. |
| Sodium hydroxide | | | q.s. | q.s. | q.s. | q.s. |
| Purified water | | | q.s. | q.s. | q.s. | q.s. |
| pH | | | 7.5 | 7.5 | 7.5 | 7.5 |
| Osmotic pressure mOsm | | | 260 | 260 | 260 | 260 |
| Residual | 40° C. 20 days | Glass | 100.6 | 100.0 | 100.1 | 99.6 |
| ratio | | PET | 100.8 | 100.4 | 99.5 | 99.1 |
| (%) | 50° C. 20 days | Glass | 100.3 | 100.1 | 99.3 | 98.9 |
| | | PET | 103.0 | 102.4 | 99.6 | 99.4 |
| | 60° C. 10 days | Glass | 102.2 | 102.4 | 101.7 | 98.3 |
| | | PET | 100.5 | 100.3 | 97.2 | 96.9 |

As is apparent from Tables 1 to 3, the addition of the fat-soluble antioxidant, BHT, greatly reduced the loss of the GGA content. The GGA content differed between the eye drops held by the glass container and the eye drops held by the PET container. This explains that the use of a container of some type allows BHT to reduce adsorption of GGA to the container wall.

Eye drops containing the all-trans form purified by the above method were prepared as follows. The constitutions of the eye drops are shown in Table 4 below.

Specifically, to a surfactant (polysorbate 80 and POE castor oil) warmed to 65° C., teprenone or the all-trans form, and retinol palmitate or sesame oil for Examples, were added and dissolved under stirring in a hot water bath at 65° C. for 2 minutes. Water at 65° C. was added and each buffer was added under stirring to give a homogeneous solution. The pH and osmotic pressure were adjusted with hydrochloric acid and/or sodium hydroxide. This resulting solution was filtered through a membrane filter with a pore size of 0.2 μm (bottle top filter, Thermo Fisher Scientific) to give a clear sterile eye drop.

Five mL of each of the eye drops was poured into various plastic or glass containers (volume: 10 to 15 mL) with a glass volumetric pipette and the containers were sealed. The container materials and the volume are shown in Table 5 below. For these eye drops, the stability test was performed by leaving them to stand in the upright position at 40° C., 75% RH for 2 hours. The all-trans form content (g/100 mL) in each of the eye drops was quantified under the HPLC conditions described above immediately after the production and after being left to stand for a predetermined period of time, and the residual ratio (%) was calculated.

The results are shown in Table 4.

TABLE 4

| | | | | | Example | Comparative | Comparative |
|---|---|---|---|---|---|---|---|
| g/100 mL | | Example 7 | Example 8 | Example 9 | 10 | Example 7 | Example 8 |
| All-trans form | | 0.05 | 0.05 | 0.05 | 0.05 | 0.05 | 0.05 |
| Sodium dihydrogen | | 0.30 | — | 0.30 | — | 0.30 | — |
| phosphate dihydrate | | | | | | | |
| Disodium hydrogen | | 3.20 | — | 3.20 | — | 3.20 | — |
| phosphate dodecahydrate | | | | | | | |
| Boric acid | | — | 1.40 | — | 1.40 | — | 1.40 |
| Borax | | — | 0.30 | — | 0.30 | — | 0.30 |
| POE castor oil | | 0.02 | 0.02 | 0.02 | 0.02 | 0.02 | 0.02 |
| Polysorbate 80 | | 0.50 | 0.50 | 0.50 | 0.50 | 0.50 | 0.50 |
| Sesame oil | | 0.05 | 0.05 | — | — | — | — |
| Retinol palmitate | | — | — | 0.05 | 0.05 | — | — |
| Hydrochloric acid | | q.s. | q.s. | q.s. | q.s. | q.s. | q.s. |
| Sodium hydroxide | | q.s. | q.s. | q.s. | q.s. | q.s. | q.s. |
| Purified water | | q.s. | q.s. | q.s. | q.s. | q.s. | q.s. |
| pH | | 7.5 | 7.5 | 7.5 | 7.5 | 7.5 | 7.5 |
| Osmotic pressure mOsm | | 260 | 240 | 260 | 240 | 260 | 240 |
| 40° C. 2 h | GLS | 99.7 | 99.8 | 99.5 | 100.0 | 99.6 | 99.4 |
| Residual ratio | PP | 96.4 | 96.0 | 96.1 | 95.5 | 94.7 | 93.9 |
| (%) | LDPE | 92.3 | 90.1 | 92.3 | 90.6 | 89.6 | 86.5 |
| | PS | 91.5 | 88.4 | 91.8 | 89.4 | 85.8 | 83.0 |
| | PBS | 91.0 | 88.6 | 91.8 | 88.8 | 86.8 | 82.8 |

(Retinol palmitate: 1,700,000 IU/g)

TABLE 5

| Material | Abbr. | Volume and type | Manufacturer | Model number |
|---|---|---|---|---|
| Glass | GLS | 10 mL centrifuge tube | IWAKI | 8084CTF10 |
| Polypropylene | PP | 15 mL conical tube | Becton, Dickinson | 352196 |
| Low density | LDPE | 10 mL sharp-bottom tube | As One | 2-467-01 |
| polyethylene | | | | |
| Polystyrene | PS | 15 mL centrifuge tube | IWAKI | 2324-015 |
| Polybutylene | PBS | 15 mL centrifuge tube | Thermo Scientific | 366079 |
| succinate | | | | |

All the containers had a v-bottom.

As is apparent from Table 4, the addition of the fat-soluble antioxidant, sesame oil or retinol palmitate, greatly reduced the loss of the GGA content. The residual ratio of GGA varied depending on the material of the container. This explains that the use of a container of some type allows sesame oil or retinol palmitate to reduce adsorption of GGA to the container wall.

### (4) Test for Residual Ratio of GGA No. 2

Eye drops containing the all-trans form purified by the above method were prepared as follows. The constitutions of the eye drops are shown in Table 6 below.

To a surfactant (POE castor oil and POE hydrogenated castor oil 60) warmed to 65° C., the all-trans form and optionally ascorbyl tetraisopalmitate were added and dissolved under stirring in a hot water bath at 65° C. for 2 minutes. Water at 65° C. was added and each buffer was added under stirring to give a homogeneous solution. The pH and osmotic pressure were adjusted with hydrochloric acid or sodium hydroxide.

Ten mL of each of the thus obtained eye drops was poured into a 13 mL eye drop container (material: low density polyethylene or high density polyethylene; shape: the same as that of the container for Namida Rohto Dry Eye (trade name) (Rohto Pharmaceutical)) with a glass volumetric pipette and the container was sealed. The container material and the volume are shown in Table 7 below.

For these eye drops, the stability test was performed by leaving them to stand in the upright position at 40° C., 75% RH for 2 hours, 8 hours, and 24 hours. The all-trans form content (g/100 mL) in each of the eye drops was quantified by HPLC immediately after the production and after being left to stand for a predetermined period of time, and the residual ratio (%) was calculated.

The results are shown in Table 6.

TABLE 6

| | | Comparative | Example | Example |
|---|---|---|---|---|
| g/100 mL | | Example 9 | 11 | 12 |
| All-trans form | | 0.005 | 0.005 | 0.005 |
| Boric acid | | 1.300 | 1.300 | 1.300 |
| Borax | | 0.400 | 0.400 | 0.400 |
| POE castor oil | | 0.100 | 0.100 | 0.100 |
| POE hydrogenated | | 2.000 | 2.000 | 2.000 |
| castor oil 60 | | | | |
| Ascorbyl tetraisopalmitate | | — | 0.100 | 0.500 |
| Hydrochloric acid | | q.s. | q.s. | q.s. |
| Sodium hydroxide | | q.s. | q.s. | q.s. |
| Purified water | | q.s. | q.s. | q.s. |
| pH | | 7.57 | 7.61 | 7.59 |
| Osmotic pressure mOsm | | 236 | 236 | 241 |
| 40° C. 2 h | LDPE-ED | 94.8 | 97.1 | — |
| Residual ratio | HDPE-ED | 96.6 | 98.3 | 103.0 |
| (%) | | | | |
| 40° C. 8 h | LDPE-ED | 85.6 | 89.2 | — |
| Residual ratio | HDPE-ED | 89.5 | 92.2 | 97.7 |
| (%) | | | | |
| 40° C. 24 h | LDPE-ED | 70.7 | 75.8 | — |
| Residual ratio | HDPE-ED | 78.1 | 82.9 | 91.4 |
| (%) | | | | |

TABLE 7

| Material | Abbr. | Volume and type |
|---|---|---|
| Low density polyethylene | LDPE-ED | 13 mL eye drop container |
| High density polyethylene | HDPE-ED | 13 mL eye drop container |

As is apparent from Table 6, ascorbyl tetraisopalmitate reduced adsorption of GGA to the wall of the containers in a dose-dependent manner.

### INDUSTRIAL APPLICABILITY

The ophthalmic composition of the present invention, in which adsorption of GGA to the wall of a container is remarkably reduced, is very useful in practice.

1-9. (canceled)

10. A method of reducing loss of geranylgeranylacetone, comprising:
a step of making an ophthalmic composition comprising geranylgeranylacetone and a fat-soluble antioxidant, and
a step of holding the ophthalmic composition in an ophthalmic container for at least 8 hours,
thereby reducing adsorption of geranylgeranylacetone to a wall of the ophthalmic container.

11. The method according to claim 10, wherein the ophthalmic composition is in the form of a liquid, a fluid, a gel or a semi-solid.

12. The method according to claim 10, wherein the fat-soluble antioxidant content in the ophthalmic composition is 0.0001 to 100 parts by weight relative to 1 part by weight of the geranylgeranylacetone.

13. The method according to claim 10, wherein the fat-soluble antioxidant content in the ophthalmic composition is 0.00001 to 10% by weight relative to the total amount of the composition.

14. The method according to claim 10, wherein the geranylgeranylacetone content in the ophthalmic composition is 0.00001 to 10% by weight relative to the total amount of the composition.

15. The method according to claim 10, wherein the ophthalmic composition is an eye drop, an eye wash, a contact lens-wearing solution, a contact lens solution, a preservative for a harvested corneal tissue for transplantation, an irrigating solution for surgery, an ophthalmic ointment or an intraocular injection.

16. The method according to claim 10, wherein the pH of the ophthalmic composition is from 4 to 9.

17. The method according to claim 10, wherein the ophthalmic composition further comprises a buffering agent.

18. The method according to claim 10, wherein the ophthalmic composition is an aqueous composition or an oil composition.
